(19) United States
(12) Patent Application Publication
(10) Pub. No.: US 2004/0215155 A1
(43) Pub. Date: Oct. 28, 2004

(54) Wound irrigator

(76) Inventors: Timothy R. Wolfe, Salt Lake City, UT (US); Marshall T. Denton, Salt Lake City, UT (US); Perry W. Croll, Salt Lake City, UT (US); Mark A. Christensen, Salt Lake City, UT (US)

Correspondence Address: TRASK BRITT, P.O. BOX 2550, SALT LAKE CITY, UT, 84110, US

(21) Appl. No.: 10/422,919

(22) Filed: Apr. 23, 2003

(51) Int. Cl. A61M 35/00

(52) U.S. Cl. 604/289

(57) ABSTRACT

A wound irrigation apparatus including a disposable portion associated with a discharged irrigation stream and a reusable fluid dispensing portion. One embodiment combines a conventional disposable splash shield with a shroud operable as a second splash shield to further protect (for reuse) a flow control device and its fluid supply conduit from splash-back of irrigating fluid. An exemplary shroud can be formed from a plastic bag. The shroud generally is used with a check valve forming a fluid path from the flow control device to a discharge nozzle exterior to the shroud. A preferred check valve may be embodied as a LUER™ joint spacer, and is structured to form a fluid resistant connection in engagement with a shroud by piercing the distal end of the check valve through a membrane wall of the shroud for reception of that distal end in a hub of a splash shield.

### TECHNICAL FIELD

The invention relates to apparatus operable to irrigate wounds. It particularly relates to wound irrigating apparatus arranged to permit reuse of certain components to reduce costs and reduce clinician time associated with an irrigation procedure.

### BACKGROUND

A conventional wound irrigation procedure employs a syringe to direct a stream of sterile irrigation fluid through a bore representative of an approximately 19 gage needle for impingement of the stream onto the wound or irrigation site. When used as a fluid pump during a wound irrigation procedure, a syringe is typically operated to generate about 8 to 15 psi in the irrigation fluid. A complete irrigation procedure may require about one liter of fluid to be squirted onto the wound. Since most syringes lack a one liter capacity, the syringe requires recurring refilling. Fluid from a source may be plumbed to the syringe in combination with a double check valve arrangement to enable convenient cyclic operation of the syringe as a pump.

One arrangement of a syringe in combination with a double check valve to pump fluid from a source is disclosed by Stamler, in U.S. Pat. No. 5,860,947. An alternative fluid pumping arrangement using a trigger actuated hand pump is disclosed in U.S. Pat. No. 4,892,526, to Reese. A third arrangement for delivering irrigation fluid to a wound is disclosed by Witt et al. in U.S. Pat. No. 5,071,104. Witt et al. disclose a container to hold irrigation fluid, and a device to pressurize the container to cause fluid flow.

Splash guards for use in irrigation procedures are well known. A splash guard is often connected to a discharge end of the syringe to substantially contain splash-back droplets of the irrigation fluid. Exemplary splash shields forming a distally directed, substantially cup shape are disclosed in U.S. Pat. Nos. 4,769,003, Des. 344,133, and Des. 345,016, all to Stamler; U.S. Pat. No. 4,898,588 to Roberts; U.S. Pat. No. 5,224,940 to Dann et al.; U.S. Pat. No. 5,496,290 to Ackerman; and U.S. Pat. No. 5,941,859 to Lerman. Lampropoulos et al. disclose a splash shield that is substantially planar in U.S. Pat. Nos. 6,050,982 and 6,093,182. Even when a splash shield is employed, a significant risk exists that splash-back from the discharge stream will contact the syringe used to pump the irrigation fluid. Naturally, the contaminated syringe must then be disposed of in a proper container.

Syringes are old in the art, and are well known. Desirable syringes for use as an irrigation pump include syringes of the type known as self-filling syringes. Such syringes are adapted to bias the plunger toward a “full” position with respect to the syringe body. However, such syringes are prohibitively costly, due to the biasing element and additional gripping structure, for single-use-and-discard applications, such as in a typical wound irrigation procedure. An alternative and higher capacity syringe is adapted to have a longer and larger diameter bore, with a plunger forming a transverse handle that can be gripped by a user's hand and palm. Such a higher capacity syringe requires less refilling, and generally is operated using both of a user's hands. However, such a higher capacity syringe is also prohibitively expensive for the single-use-and-discard cycle of a typical irrigation procedure.

It would be an advance to provide an irrigation apparatus that increases efficacy of an irrigation procedure. One advance would alleviate the tedious manual refilling of a syringe used as a fluid pump. A further advance would permit reuse of certain components of the irrigation assembly, to amortize the cost of a portion of the assembly over a plurality of procedures.

### BRIEF SUMMARY OF THE INVENTION

An irrigation apparatus typically combining a conventional distally directed and disposable splash shield with a shroud operable as a second, proximally directed splash shield to further protect (for reuse) a flow control device and its fluid supply conduit from splash-back of irrigating fluid. An exemplary shroud can be formed from a plastic bag. The shroud generally is used with a check valve forming a fluid path from the flow control device to a discharge nozzle located exterior to the shroud. A preferred check valve may be embodied as a Luer joint spacer, and is structured to form a fluid resistant connection in engagement with a shroud by piercing the distal end of the check valve through a membrane wall of the shroud for reception of that distal end in a hub of a splash shield.

A shroud can be arranged to define a volume in which to contain a portion of a distal end of a fluid dispensing apparatus, such as a self-filling syringe and a portion of fluid supply tubing. A shroud may be considered as a wall disposed about a perimeter of the volume and providing an aperture through which an irrigation fluid may pass. The shroud functions to resist contact between the fluid dispensing apparatus and splash-back particles of irrigation fluid. The aperture through the shroud wall desirably forms a fluid resistant connection with a penetrating conduit connectable to a discharge end of the dispensing apparatus. Generally, a check valve is included in the penetrating conduit to permit flow of irrigation fluid through the shroud in a direction toward a discharge orifice and to resist fluid flow in a reverse direction.

Certain shrouds include portions that are transparent. The wall of a typical shroud can be characterized as being a membrane. A shroud includes a proximally oriented opening typically sized to accommodate insertion of a syringe and a user's hand to protect them from contact with splash-back particles of irrigation fluid. A currently preferred shroud is formed from a plastic bag having a depth of about 14 inches, and a width of about 10 inches, when measured in a flat configuration. Such a plastic bag can form a short sleeve having a diameter of about 6 inches. Certain preferred shrouds may be formed from an envelope having a length between about eight and about thirty inches, and a width between about six and about eighteen inches, when measured in a flat configuration.

A penetrating conduit passing through a wall of the shroud extends the flow path from the flow control device toward a discharge orifice (typically comparable to a 19 gauge bore) for irrigation of a wound. A distal end the penetrating conduit desirably includes piercing structure adapted to form the aperture through the shroud. The penetrating conduit desirably includes attachment structure carried at a proximal end for connecting to a fluid dispensing apparatus. Preferably, the connection is removable to permit disposal of the penetrating conduit and shroud, and reuse of the dispensing apparatus. Typically, the penetrating conduit includes a shroud check valve operable to permit flow of irrigation fluid only in a direction from the inside to the outside of the shroud.

The invention typically includes a fluid flow control device disposed in fluid communication with the shroud check valve. The flow control device desirably is operable to effect a flow of irrigation fluid as desired. One exemplary flow control apparatus includes a syringe and a double check valve arrangement providing a first check valve disposed in a first fluid path to a fluid source and a second check valve disposed in a second fluid path to a discharge opening. Such a plumbing arrangement permits cyclic operation of the syringe without either requiring disconnection of the syringe from the fluid flow path, or manual manipulations of other valves. A second flow control apparatus can be embodied as a simple valve. In one such case, the valve is operable to occlude fluid flow between a pressurized fluid source and a discharge from the wound irrigation device.

One pressurized fluid source within contemplation is tap water from a utility infrastructure. A second pressurized fluid source may be embodied as a portable chamber adapted to compress a fluid-filled container, such as a one liter bag of sterile saline, or a refillable fluid bag. In arrangements including pressurized fluid sources, it is sometimes desirable to include a flow meter configured and arranged to measure a quantity of irrigation fluid flow. Alternatively, structure may be arranged to indicate an amount of fluid remaining in the fluid source. A “pig-tail” is provided in certain preferred embodiments to permit incremental draining of a bag during a plurality of irrigation procedures. In such case, a discard tube section connects a discharge orifice to the pig-tail.

Also, a pressure reducing apparatus can be disposed in the fluid path between the pressurized fluid source and the discharge fluid stream from the invention. The pressure reducing apparatus can reduce a discharge pressure of the irrigation fluid to a desired pressure lower than a pressure of irrigation fluid at the source.

### BRIEF DESCRIPTION OF THE SEVERAL VIEWS OF THE DRAWINGS

In the drawings, which illustrate what are currently considered to be the best modes for carrying out the invention:

FIG. 1 is an illustration, partially in section, depicting a first wound irrigating assembly according to the invention;

FIG. 2 is an illustration depicting a second wound irrigating assembly according to the invention;

FIG. 3 is a view in elevation, substantially in section, taken along a major axis through the mid-plane of a first preferred check valve for use as a penetrating conduit to penetrate a shroud;

FIG. 4 is a view similar to the view illustrated in FIG. 3, of a second preferred check valve in combination with a shroud and a hub portion of a splash shield;

FIG. 5 is a view in elevation, substantially in section, taken along a major axis through the mid-plane of a third preferred check valve;

FIG. 6 is a sectional view in elevation of a double check valve arrangement beneficial for use with certain embodiments of the invention that include a syringe to pump irrigation fluid;

FIG. 7 is a view in elevation, substantially in section, of a currently preferred double check valve;

FIG. 8 is a plan view in elevation of a currently preferred wound irrigation system;

FIG. 9 is a plan view of a disposable wound irrigating portion of the system illustrated in FIG. 8;

FIG. 10 is an end view of a workable, and currently preferred, line-valve operable to control fluid flow through the device illustrated in FIG. 9; and

FIG. 11 is a side view of the line-valve of FIG. 10 installed on a section of tubing.

### DETAILED DESCRIPTION OF THE ILLUSTRATED EMBODIMENTS

For convenience, the terms “proximal” and “distal” will be used in this disclosure to define a relative orientation of structure. In relation to structure forming, or disposed adjacent to a fluid flow path, a “proximal” end will typically be closer to a fluid source than a “distal” end.

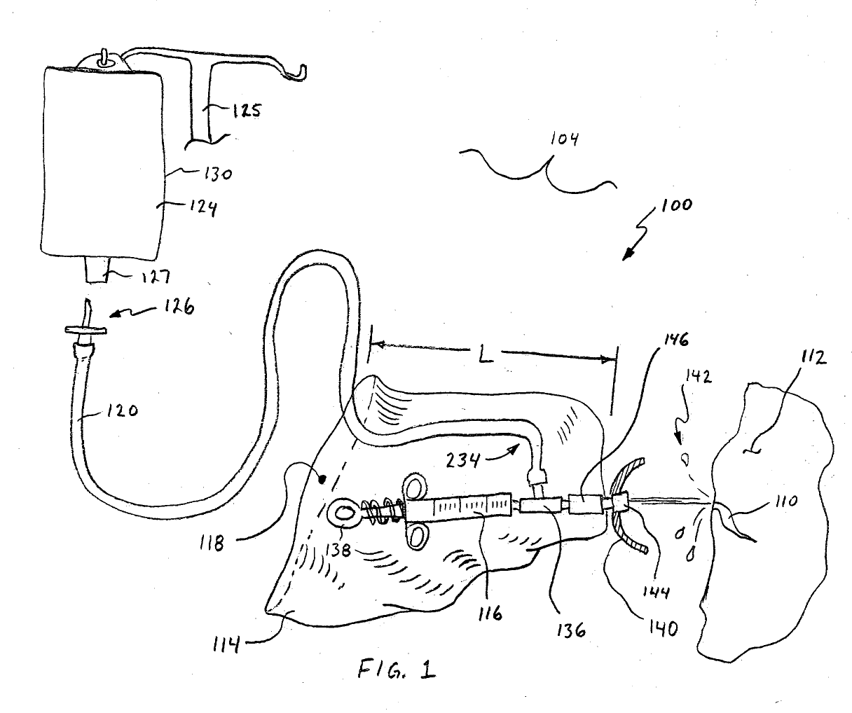

As illustrated in FIG. 1, one embodiment of the invention, generally indicated at 100, is included in an apparatus, generally indicated at 104, adapted for irrigating a wound 110, or a contaminated site on a patient 112. The invention 100 includes a shroud 114 sized to define a volume in which may be inserted a fluid flow control device, such as syringe 116, and a user's hand. Shroud 114 may be described as being proximally oriented, in that shroud 114 has an opening 118 that faces in a proximal direction. Operable shrouds 114 form a structure that may be regarded as a sleeve, enclosure, or envelope, having an opening at one end and being serviceable as a cover to shield at least a distal portion of a fluid flow control device from contamination. Preferably, the shroud 114 also protects a distal portion of a fluid supply conduit 120.

Shrouds 114 typically are formed from material providing a flexible, fluid resistant membrane. It is currently preferred for shrouds 114 to include at least a portion that is transparent to facilitate operation of a syringe 116, or other fluid flow control device. A currently preferred embodiment of a shroud 114 is made from a substantially rectangular plastic envelope or bag having a length L1 of about 14 inches, and a height H₁ (related to an opening 118, see FIGS. 1 and 2) of about 10 inches, when measured in a flat configuration. When expanded to define a volume, such a bag can form an open-ended sleeve with a diameter of about 6 inches.

The overall configuration and expanded shape of a shroud 114 is not important, as long as the shroud 114 can be disposed to be operable as a cover for certain portions of an irrigating assembly 104. A shroud 114 may be made to have any size and shape desired for particular applications. In particular, shrouds 114 do not need to be rectangular. Most desirable shrouds 114 may have a length L in a range of about 6 inches to about 30 inches, when measured in a flat, or collapsed, full length configuration. A realistic upper limit to useful shroud lengths may be determined by the length of a user's arm. A minimum height for a shroud 114 must accommodate insertion of a flow control apparatus into the shroud. It currently is preferred also to insert a user's hand into the shroud 114.

A wound irrigation apparatus 104 typically includes a conduit 120 attached to a fluid source, such as a bag 124 of saline suspended from a stand 125. One way to form a connection between conduit 120 and container 124 is by use of a spike tap, generally indicated at 126. Structure of spike 126 is pressed into fluid-tight engagement in spout 127 of bag 124. Of course, many other plumbing connections are operable to connect the conduit to a fluid supply. Certain other operable connections include screw-on adapters and press-fit, or slip-on connections.

It is currently general practice for the irrigating fluid to be a sterile fluid, although in certain instances tap water may used. In most modern communities, potable water drawn from the public utility infrastructure is sufficiently clean and low in pathogen count to enable its use as a substitute irrigation fluid. Therefore, a container 130 to hold an irrigation fluid may be refillable, and may be sized to accommodate a plurality of liters of irrigation fluid. Such a container 130 may include a plurality of volume indicating marks. Alternatively, with reference to FIG. 2, the fluid source can be the utility infrastructure in a direct connection, generally indicated at 132.

With continued reference to FIG. 1, an irrigation assembly 104 typically includes a fluid control device, such as syringe 116, operable to permit fluid flow in conduit 120. Illustrated syringe 116 is connected in fluid communication with conduit 120 through a double check valve 136. Double check valve 136 permits cyclic discharge of fluid from the syringe 116 and automatic refilling of the syringe 116 without requiring manual actuation of the valve 136, or disconnection of the syringe from the fluid circuit.

While any sort of syringe may be used in the irrigation device 104, it is currently preferred to use a self-filling syringe 116, as illustrated. A self-filling syringe 116 has a biasing element adapted to return the plunger 138 from an “empty” or compressed position to a “filled” or extended position. An alternative preferred, and generally larger volume, syringe (not illustrated) provides a transverse handle that can be gripped in a user's palm. Such a syringe is typically operated with both hands; one hand gripping the syringe body and one hand operating the plunger.

An irrigation assembly 104 generally is considered to be a use-once-and-dispose-of device. Use of a preferred flow control device, such as self-filling syringe 116, can facilitate performance of an irrigation procedure by reducing repetitive stress on the health practitioner, or irrigator. However, such a flow control device can be a costly component of assembly 104. Therefore, it is desirable to reuse certain flow control devices, such as a self-filling syringe 116, for several irrigation procedures to amortize the cost of the flow control device over several patients or procedures. Desirably, the proximal portion of the assembly 104, from the spike tap to the flow control device 116 and double check valve 136, is salvageable for reuse during multiple irrigation procedures.

Still with reference to FIG. 1, irrigation assembly 104 typically includes a distally directed splash shield 140, illustrated in cross-section. Splash shield 140 is typically held in close proximity to the surface of the site being irrigated. Certain preferred splash shields 140 are adapted to permit holding a perimeter edge in contact with a surface of the patient. Shields, such as splash shield 140, are used to resist contact between the irrigator and particles of irrigation fluid, generally indicated at 142. However, fluid particles 142 can sometimes escape confinement and impinge onto portions of the irrigating apparatus 104. Impinging particles 142 would compromise sterility of the apparatus 104, prohibiting reuse of contaminated portions. A shroud 114 can effectively intercept such particles 142, permitting reuse of certain portions of the irrigating apparatus 104.

It is currently preferred to attach splash shield 140 to a conduit structure that pierces a wall of the shroud 114. Commonly, hub 144 of shield 140 is affixed to a distal portion of a check valve 146. Illustrated check valve 146 is disposed to permit fluid flow through a conduit in a direction from valve 136 toward a discharge orifice, and to resist fluid flow in a reverse direction. It is currently preferred to incorporate a check valve 146 as part of a unitary structure forming the conduit that pierces a wall of the shroud 114. However, it is within contemplation alternatively to attach a separate check valve component in fluid communication with a wall-piercing conduit. In any case, it is desirable for the check valve 146 to be removable from engagement with a fluid flow control device, such as syringe 116, to permit disposal of the valve 146 along with a shroud 114 and shield 140.

FIG. 2 illustrates an alternative arrangement of irrigation equipment, generally indicated at 150. The assembly 150 is adapted to use tap water as a pressurized fluid source in a direct connection 132. A flow path from the fluid source to a discharge orifice can be formed from sections of conduit, including disposable tubing sections 151A and 151B. A pressure reducer 152 may be included in the flow path between connection 132 and a discharge of the irrigation apparatus 150. A flow indicating device, such as in-line flow meter 154, desirably is also included in an apparatus 150. Illustrated flow meter 154 includes a resetable display screen 156 to indicate the quantity of fluid that has passed through the meter to assist an irrigator in satisfaction of volumetric requirements of an irrigation procedure.

Flow control device 158 illustrated in FIG. 2 is disposed to control flow of the irrigation fluid for discharge through splash shield 160. Device 158 functions as a valve, and includes an actuator 159 operable to permit fluid flow through the device for discharge of the fluid through shield 160. Preferably, flow control device 158 is protected in a shroud 114 during irrigation procedures, to help maintain sufficient sterility of the device 158 to permit its reuse. Preferred devices 158 are also separately sterilizable to permit their reuse. A flow control device 158 may include an integral flow meter to measure flow of irrigation fluids and arranged for convenient observation by the irrigator. Furthermore, device 158 may be fashioned in any convenient form, including as a pistol grip, or the illustrated substantially in-line configuration. A distal end of flow control device 158 desirably caries connection structure arranged to form a slip, Luer-type, or threaded connection with a proximal end of fluid path extension structure, such as conduit 162.

Splash shield 160 typically is attached to a conduit section 162 arranged to pass through a wall of the shroud 114. Conduit 162 desirably carries a check valve operable to resist fluid flow in a direction from the exterior toward the interior of the shroud 114. Furthermore, conduit 162 preferably is removable from fluid communication with the device 158, to permit disposal of the conduit 162 along with a shroud 114 and splash shield 160.

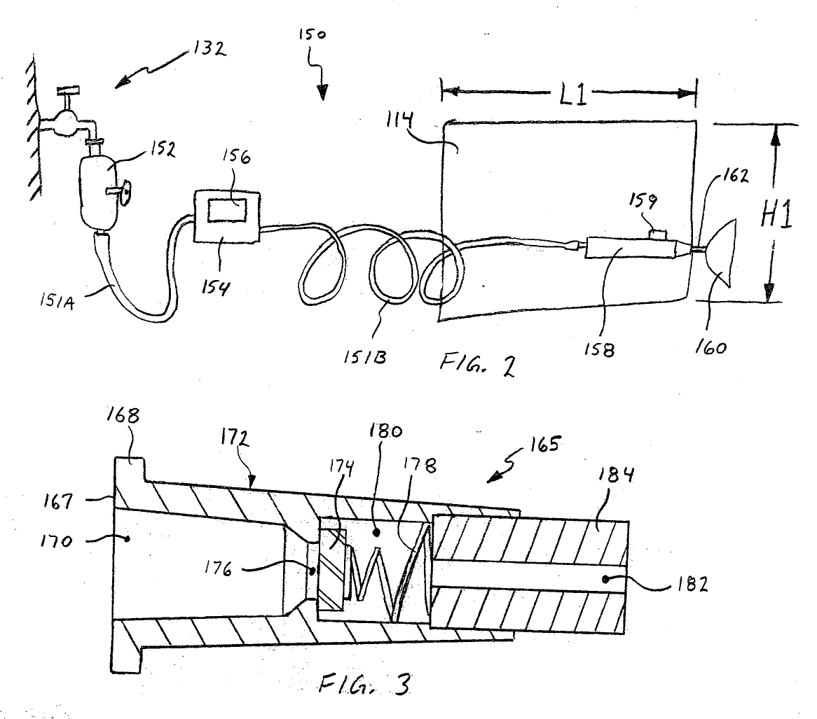

FIG. 3 illustrates one currently preferred embodiment of a conduit 160, indicated generally at 165. As illustrated, conduit 165 may be characterized also as being a check valve. Conduit 165 may also be regarded as a joint spacer, or extension. In an assembly 104 illustrated in FIG. 1, conduit 165 forms a syringe-tip extension. A check valve desirably is located between fastening structure arranged on proximal and distal ends of the conduit 165. A proximal end 167 of conduit 165 carries fastening structure, such as illustrated male thread 168 and tapered bore 170 in a conventional arrangement as part of a Luer-type joint. Bore 170 can form a slip fit to an inserted tip of a syringe or other flow control device. Structure carried on a distal end of conduit 165, such as surface 172, typically is adapted to form a slip-type fit with a hub of a splash shield, such as shield 140. Surface 172 may be arranged as a portion of a Luer-type joint structure.

A check valve is formed by resilient element 174 being biased into engagement over aperture 176 to resist fluid flow in one direction, and to permit fluid flow in the reverse direction. A biasing element, such as spring 178, maintains seal element 174 in obstructing engagement until overcome by fluid pressure in bore 170. Irrigation fluid of sufficiently high pressure in bore 170 causes seal element 174 to separate from engagement with aperture 176, and thereby permits fluid flow in a distal direction. Seal member 174 resists fluid flow in the reverse direction, and even seals more tightly when fluid pressure in conduit portion 180 is higher than fluid pressure in bore 170.

Seal member 174, illustrated in FIG. 3, desirably is shaped in harmony with a cross-section shape of bore 180 to permit irrigation fluid to flow past an edge, or perimeter, of the seal member 174. As an example, one workable arrangement incorporates a lumen or bore 180, having a circular cross-section, paired with a seal 174 having a hexagonal cross-section. Spring 178 may have a plurality of diameters, as illustrated, to reliably bias seal element 174 into proper engagement over aperture 176. Ends of spring 178 may alternatively be configured to form a reliable interface with the seal 174, to resist tipping or rotation of the seal 174.

A discharge nozzle can conveniently be formed as a bore, or aperture 182, passing through a cylindrical plug or section of tubing 184. Conventionally, aperture 182 is sized congruent to the discharge aperture provided by a 19 gauge needle. It currently is preferred for distal structure of conduit 165 to be structured effective as a piercing point, to enable punch-forming an aperture through a shroud 114 at a desired location. Sometimes, a “starter hole” may be provided in a shroud 114. In any case, structure, such as surface 172, desirably is provided operable to form a fluid resistant attachment with a shroud 114.

FIG. 4 illustrates one workable arrangement for using a shroud 114 in combination with a splash shield 160. A check valve, generally indicated at 190, is disposed in piercing engagement through shroud 114. Desirably, check valve 190 is configured to permit piercing, punching, or poking, a distal portion of the valve 190 through shroud 114 at a desired location. Check valve 190 then forms a conduit arranged to permit flow of irrigation fluid through the shroud 114 in a direction toward a discharge orifice 192, and to resist fluid flow in a reverse direction.

Shroud 114 typically forms a fluid resistant coupling to conduit 190 by engaging surface 194. It is within contemplation also to provide backing structure (not illustrated) as a stop to resist further penetration of conduit 190 through the shroud 114. The shroud 114 can be effectively pinched, or compressed, between such backer structure and a pressed-on hub 144. Such an arrangement can form an operable fluid resistant joint between a shroud and a piercing conduit.

It should be noted that a splash shield 160 may include a check valve to resist fluid flow in a direction other than for irrigating. In such case, conduit 190 may be provided without a check valve arrangement. A splash shield 160 may optionally include a discharge orifice operable to form a desired discharge irrigation fluid stream. In such case, conduit 190 may be configured simply to pierce the shroud 114 for attachment to the hub 144. Certain splash shields may even provide a proximal conduit section operable to pierce the shroud 114 for connection to a fluid source.

Similar to valve 165, check valve 190 includes a resilient seal element 174 over an entrance orifice 176. Biasing element 194 is embodied as a spring having a substantially uniform diameter. An orientation of the spring 194 is maintained by its conformance to a diameter of lumen 196. Spring 194 is therefore oriented to engage seal 174 in a line-of-action to resist tipping or cocking of the seal 174.

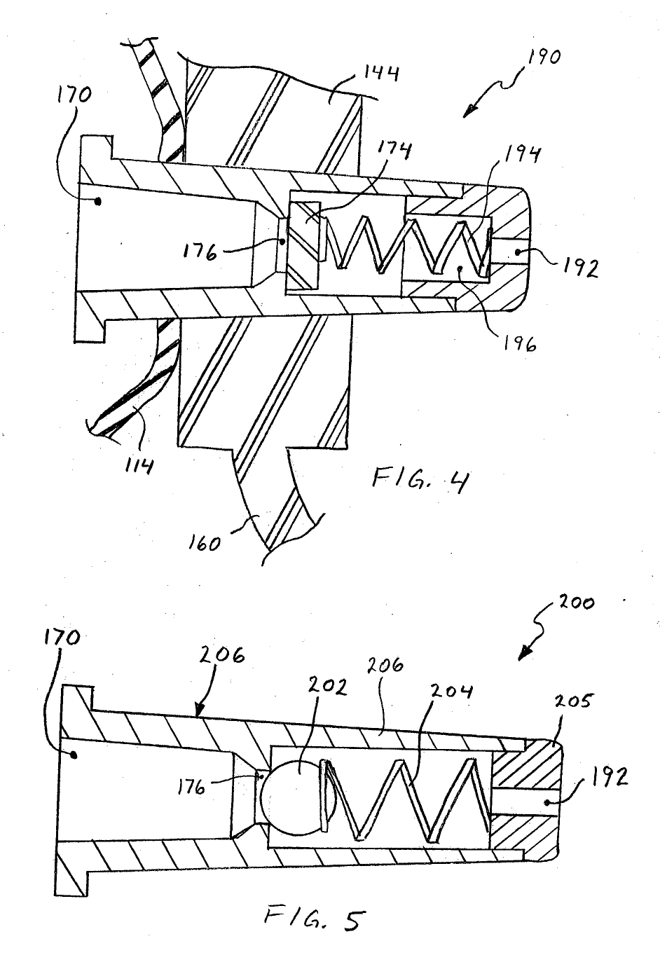

FIG. 5 illustrates a third embodiment of a conduit, generally indicated at 200, adapted to pierce a shroud 114. Conduit 200 can place a fluid control device in fluid communication with a discharge orifice 192 for irrigating a patient. In most typical use, the conduit 200 holds a splash shield engaged to conical surface 206. As illustrated, conduit 200 carries a check valve portion including a sphere 202 biased into engagement over orifice 176 by spring 204.

Certain constituent components forming conduits, such as conduit 200, are typically formed by injection molding from medical grade plastic materials. Components, such as tip 205, may be adhesively bonded, or welded to a conduit body 206. Seal members, such as seal 202 or seal 174, may be formed from a suitably soft and resilient material, such as urethane or silicone rubber. Seal member 174 preferably is formed from short sections cut from an extruded silicone rod having a hexagonal cross-section. Biasing elements, such as springs 194 and 204, can be plugs or sections of resilient material, such as rubber, or can be coil springs, as illustrated.

FIG. 6 illustrates a favorable plumbing arrangement with check valves. 210 and 211 arranged to facilitate operation of a cyclic pump, such as a syringe 116 in preferred embodiments of the invention 100. A lumen 212 of conduit 214 is placed into fluid communication with a fluid source. Check valve 210 is placed in the flow path to permit fluid flow from the source only toward a “T” fitting 216. Lumen 220, which could represent the fillable volume of a syringe, may be regarded as a staging zone. Pressure is cycled at the staging zone to draw fluid from the source, and expel that fluid through check valve 211, and then through lumen 224. Lumen 224 can then transport irrigation fluid to a discharge nozzle, such as aperture 192 (see FIG. 4 or 5). The illustrated arrangement of check valves 210 and 211 permit cyclic operation of a syringe without requiring the syringe to be disconnected for refilling. Furthermore, the check valves operate under the influence of pressure caused by the syringe, so their operation is automatic. That is, no valves need be operated manually between strokes of the syringe's plunger.

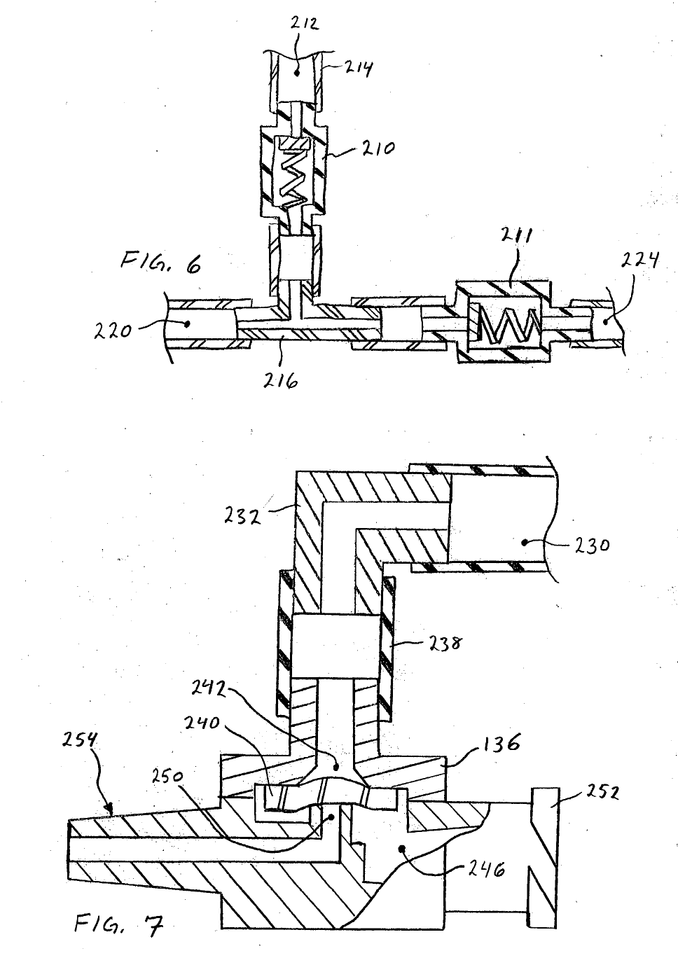

FIG. 7 represents a plumbing arrangement equivalent to that illustrated in FIG. 6, but in a low cost, unobtrusive, packaging arrangement currently preferred for use with the invention 100. Lumen 230 is placed into fluid communication with a fluid source, such as a bag 124. An angle adapter 232 sometimes is incorporated, at the area generally indicated at 234 in FIG. 1, to facilitate placing a syringe 116 into a shroud 114. Angle fitting 232, if present, may then be connected with tubing 238 to double-check valve 136.

Double-check valve 136 includes a resilient seal member 240 that is self-biased into covering engagement over an opening in lumen 242. Fluid flows past the edges of seal 240 when under a higher pressure in lumen 242 than fluid in staging area 246. Flow in the reverse direction is resisted by seal member 240, which reseats to cover the opening to lumen 242 when the pressure in lumen 242 is reduced. A buildup of pressure in staging lumen 246 simply presses seal 240 into tighter engagement to further resist fluid flow in a reverse direction through lumen 242. However, the pressurized fluid from lumen 246 forces the seal 240 away from sealing engagement over aperture 250, permitting fluid flow through aperture 250 to exit the valve 136.

Attach structure, such as male thread 252 and a tapered bore 246, carried on a proximal end of valve 136 permit convenient attachment of valve 136 to a syringe 116. Such illustrated proximal attach structure is fashioned as a portion of a Luer-type joint. Structure carried on a distal portion of valve 136, such as conical surface 254, may interface for reception in a slip fit within a cooperating shaped bore. It is currently preferred for distal structure 254 to engage in fluid tight communication with a conduit, such as bore 170 of check valve 190.

FIG. 8 illustrates a wound irrigation system according to the invention, generally indicated at 260, incorporating a reusable pressurized fluid source. Such a system 260 relieves an irrigator of the tedious chore of cyclic actuation of a syringe to effect a discharge of irrigating fluid. The fluid source can include a portable chamber 262 adapted to compress a fluid-filled container 264, such as a commercially available one liter bag of sterile saline (illustrated in phantom outline). Alternatively, bag 264 could be a refillable fluid bag to hold tap water, or any other suitable irrigation fluid. Desirably, bag 264 has a fluid capacity in excess of about one-quarter of a liter to permit discharge of an uninterrupted irrigation stream onto at least one wound. The quantity of fluid provided by a bag 264 desirably will satisfy the irrigation requirement for at least one wound, and permit an irrigation procedure to be accomplished with a steady stream of fluid.

An operable chamber 262 can be constructed to compress a bag 264 using a gravity actuated weight, or a mechanical or pneumatic spring. As illustrated, chamber 262 houses a resilient bladder 266 (illustrated in dashed outline) that can be inflated with a gas to compress bag 262. A workable bladder 266 can be fashioned from a bicycle inner tube. One way to inflate a bladder 266 is with an air pump 268 and tubing 269. A compressed gas source may also be used to inflate a bladder 266. A pump 268 and chamber 262 can conveniently be installed on a stand 270 to form a portable wound irrigation system 260.

Since the desired dynamic pressure of an irrigation stream of fluid ranges between about 8 and 15 psi, the illustrated compression chamber 262 desirably has a volume larger than about twice the volume of a bag 264. Such an arrangement can maintain the dynamic pressure of the discharge fluid stream in the desired range without requiring addition of gas to the bladder 266 during an irrigation procedure. Similarly, in the case of a chamber 262 adapted to compress a bag 264 under influence of a mechanical spring, the effective spring length desirably is greater than about twice the uncompressed length of an installed bag 264.

A compression chamber 262 desirably includes an access panel or door 274 sized to permit loading a bag 264 into the interior of the chamber 262. A desirable door 274 is transparent to permit observation of an amount of fluid remaining in bag 264. Alternatively, some structure desirably is provided to provide an indication of the amount of fluid present in a bag 264. For example, a flow meter can be adapted to provide such feedback to an irrigator. Piston structure (not illustrated) pressing onto a bag 264 can be arranged to form a gage to indicate the remaining volume in a bag 264.

When a compression chamber 262 is adapted to compress a bag 264 using a pneumatic spring, such as bladder 266, a pressure gage 276 desirably is adapted to show the pressure inside the bladder 266. The gage 276 may be calibrated, to account for pressure losses in the chamber and plumbing conduits, directly to indicate the dynamic pressure of the discharge fluid stream. Furthermore, a dump valve 278 typically is included to permit removal of a portion of gas from a bladder 266 to facilitate installation of a bag 264.

Still with reference to FIG. 8, the invention 260 includes a disposable conduit or discard tube 284 having a length sized to locate the chamber 262 away from contact by splashback particles of discharged irrigation fluid. Conduit 284 can be discarded after a single use, while fluid source 262 can be reused multiple times. A proximal end of the conduit 284 is adapted for removable fluid-flow connection to the pressurized fluid-holding bag 264 to transport irrigation fluid to a remote discharge orifice for discharge of irrigation fluid as a stream 286. Sometimes, a distally directed splash shield 288 is used to help confine splashback particle of irrigation fluid. A first flow control device 290 is provided to permit, or to resist, flow of the irrigation fluid through a lumen of conduit 284.

Sometimes a conduit 284 further includes a Luer connection device 294 for making an intermediate connection to other conduit structure. One such conduit structure within contemplation is a pig-tail 296 that can remain attached to a partially drained bag 264. In such case, a second fluid flow control device 298 may be disposed on pig-tail 296 to resist loss of irrigation fluid from bag 264 between irrigation procedures.

With reference now to FIG. 9, a proximal end of conduit 284 is typically associated with a spike, generally indicated at 300, for forming a fluid connection with a port of bag 264. If a pig-tail 296 is included, a distal end of pig-tail 296 carries a first portion 302 of luer connection structure 294. A proximal end of conduit 284 carries a second portion 304 of luer connection structure 294. Luer connection structure 294 typically is arranged as a luer-locking type joint. However, any structure operable to connect two lengths of conduit is also workable as a luer connection structure within the ambit of this disclosure, including a hollow spike or plug carried by one conduit for force-fit reception in a lumen of the other conduit.

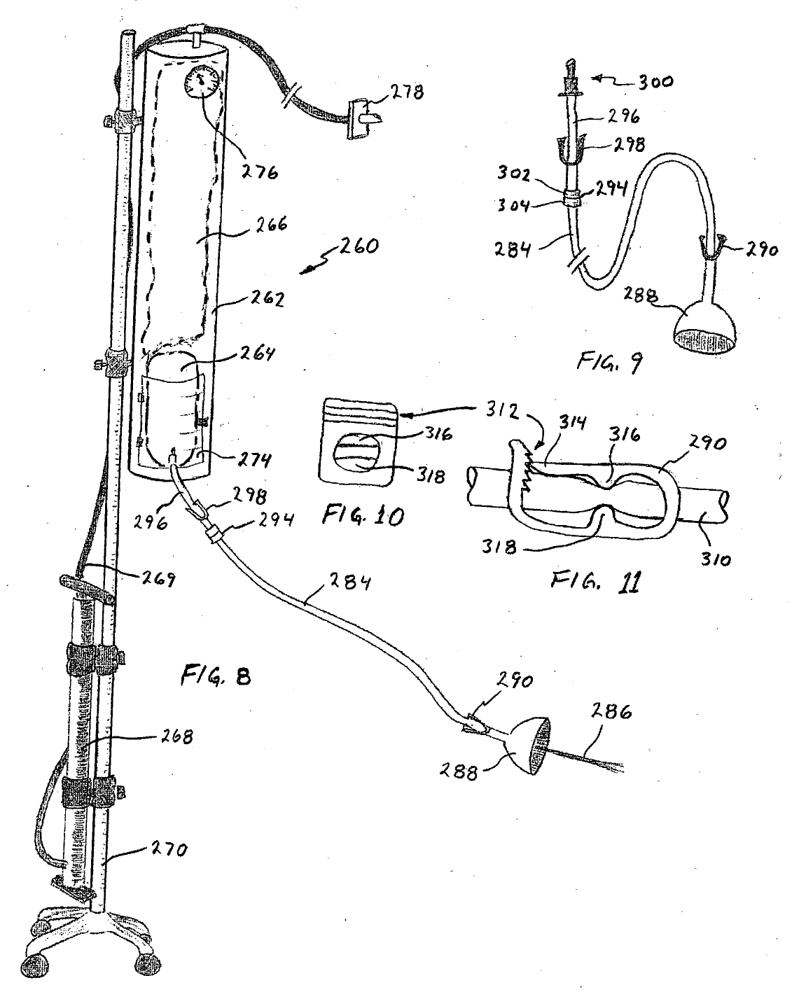

Details of construction of a currently preferred, and inexpensive, fluid flow control device 290 are illustrated in FIGS. 10 and 11. Device 290 forms a simple valve that may be opened or closed to permit or resist fluid flow through a tube 310 on which the device 290 is installed. A rack of teeth, generally indicated at 312, are adapted to hold lever arm 314 at a desired clamping position. Top jaw 316 and bottom jaw 318 are thereby correspondingly forced into engagement with walls of tube 310. Lever arm 314 may be disengaged from rack 312 to effect a flow of pressurized irrigation fluid through a lumen of conduit 310. Of course, any other device operable to control flow of irrigation fluid through a lumen of conduit 284 would also be workable in the instant invention.

What is claimed is:

1. A splash guard to protect certain dispensing apparatus of a wound irrigator assembly from splashback contamination, the guard comprising:
a shroud defining a volume in which to contain a portion of a distal end of said dispensing apparatus, said shroud comprising a wall disposed about a perimeter of said volume and providing an aperture through which an irrigation fluid may pass, said shroud further being operable to resist contact between said portion and splashback particles of said irrigation fluid.

2. The splash guard of claim 1, wherein:
said aperture of said wall is configured and arranged in harmony with structure carried at a discharge end of said dispensing apparatus to form a fluid resistant connection therebetween.

3. The splash guard of claim 1, in combination with:
a check valve disposed to effect fluid flow through a conduit disposed in piercing engagement through said aperture, said check valve being arranged to permit flow of said irrigation fluid through said shroud in a direction toward a discharge orifice and to resist fluid flow in a reverse direction.

4. The splash guard combination of claim 3, wherein:
said shroud comprises a transparent membrane; and
a distal end of structure associated with said check valve comprises piercing structure, said piercing structure being adapted to form said aperture through said membrane, said check valve comprising attachment structure configured and arranged to be carried at a distal end of said dispensing apparatus.

5. The splash guard combination of claim 3, wherein:
said check valve is removable from engagement with said dispensing apparatus to permit removal and disposal of said check valve with said shroud.

6. The splash guard of claim 1, in combination with a distally directed splash shield connected to receive fluid flow from a check valve associated with said shroud so as to permit irrigation of a wound under additional splash protection provided by said shield.

7. The splash guard of claim 1, further comprising:
a fluid flow control apparatus disposed in fluid communication with a check valve associated with said shroud, said flow control apparatus being operable to effect a flow of said irrigation fluid.

8. The splash guard of claim 7, wherein:
said shroud comprises a proximally oriented opening sized to accommodate insertion therethrough of said portion and a user's hand to dispose said portion and said user's hand inside said volume so as to effect protection of said portion and said user's hand from contact with said splashback particles.

9. The splash guard of claim 8, further comprising:
a flow control apparatus comprising:
a syringe, and;
a double check valve arrangement providing a first check valve disposed in a first fluid path to a fluid source and a second check valve disposed in a second fluid path to a discharge.

10. The splash guard of claim 8, further comprising:
a flow control apparatus comprising a valve, said valve being disposed to occlude fluid flow between a pressurized fluid source and said discharge end.

11. The splash guard of claim 10, further comprising:
a flow meter configured and arranged to measure a quantity of irrigation fluid flow for discharge of said irrigation fluid through said discharge end.

12. The splash guard of claim 11, further comprising:
a pressure reducing apparatus disposed in a fluid path between said pressurized fluid source and said dispensing end, said pressure reducing apparatus being effective to reduce a discharge pressure of said irrigation fluid at said discharge end to a pressure lower than a pressure of irrigation fluid at said source.

13. An apparatus for irrigating a wound, comprising:
a first conduit defining a first stretch of a fluid path between a fluid source and a discharge orifice, said first conduit being adapted at a first end to receive fluid from said fluid source, and adapted at a second end for fluid communication to a fluid flow regulator;
a said fluid flow regulator disposed in said fluid path and operable to permit fluid flow through said first stretch of said fluid path;
a bag sized to provide a volume in which to receive a distal portion of said flow regulator, said bag being operable to resist contact between said distal portion and splash-back particles of said irrigation fluid; and
a second conduit defining a second stretch of said fluid path, said second conduit passing through a wall of said bag to dispose said orifice for fluid discharge exterior to said bag.

14. The apparatus of claim 13, further comprising:
a distally directed splash shield comprising a hub adapted for connection to a distal portion of said second conduit.

15. The apparatus of claim 13, further comprising:
a check valve disposed in said second stretch of said fluid path and operable to permit fluid flow in a direction from said flow regulator toward a discharge orifice and to resist flow in a reverse direction, a proximal end of said second conduit being adapted for removable connection to structure provided by said flow regulator.

16. The apparatus of claim 15, further comprising:
a distally directed splash shield comprising a hub adapted to couple with structure carried on a distal portion of said second conduit.

17. The apparatus of claim 16, wherein:
said orifice is carried at a distal end of said second conduit.

18. The apparatus of claim 16, wherein:
piercing structure carried at a distal end of said second conduit is operable to form an opening through said wall of said bag; and
sealing structure carried near said distal end of said second conduit is operable to form a fluid resistant coupling between said second conduit and said bag.

19. The apparatus of claim 13, said bag comprising a transparent membrane.

20. The apparatus of claim 19, said bag comprising an envelope having a length between about eight and about thirty inches and a width between about six and about eighteen inches, when measured in a flat configuration.

21. The apparatus of claim 20, said bag comprising aplastic envelope having a length of approximately fourteen inches and a width of approximately ten inches, when measured in a flat configuration.

22. An apparatus for irrigating a wound, comprising:
a source of a fluid, said fluid being under pressure and having an initial volume in excess of about one-quarter of a liter;
a disposable conduit having a length, between a first end and a second end, sufficient in size to permit locating said source away from contact by splash-back particles of fluid during an irrigation procedure, a first end of said conduit being adapted for removable connection to said source so as to transport fluid from said source through a lumen in said conduit;
a flow control device disposed at a location along said length and operable to resist flow of said fluid through said lumen; and
a discharge orifice associated with said second end and configured and arranged to discharge said fluid as a stream for impingement of said stream onto a wound.

23. The apparatus of claim 22, further comprising:
a distally directed splash shield associated with said discharge orifice to reduce contact of splash-back particles of said fluid onto an irrigator during an irrigation procedure.

24. The apparatus of claim 22, said source comprising:
a chamber structured and arranged to compress a bag that contains said fluid.

25. The apparatus of claim 24, wherein:
said chamber comprises a housing defining a first volume in which is held a pneumatic bladder and said bag, said bag defining a second volume in which initially to hold said fluid, said first volume being greater in size than said second volume by a factor of about two.

26. The apparatus of claim 25, further comprising:
a pressure indicating device adapted to indicate a pressure exerted by said bladder;
an injection port in fluid communication with said bladder, through which port may be injected a gas to pressurize said bladder;
a dump valve adapted to permit draining a portion of said gas from said bladder; wherein:
a wall of said chamber comprises a door sized to accommodate a bag to permit locating said bag in said first volume.

27. The apparatus of claim 24, said conduit further comprising:
a spike configured for reception in a port carried by a said bag so as to permit fluid flow from said bag toward said discharge orifice.

28. The apparatus of claim 27, said conduit further comprising:
a pig-tail adapted to permit incremental discharge of a said bag during a plurality of irrigation procedures, a proximal end of said pig-tail carrying said spike and a distal end of said pig-tail carrying a first portion of a Luer connection device;
a first fluid flow device disposable to resist fluid flow through said pig-tail;
a discard tube, a proximal end of said discard tube carrying a second portion of said Luer connection device, a distal end of said discard tube being associated with said discharge orifice; and
a second fluid flow control device disposable to resist fluid flow through said discard tube.

29. The apparatus of claim 28, wherein:
structure is provided in association with said chamber to indicate a quantity of fluid remaining in said bag.
